(19) United States
(12) Patent Application Publication
(10) Pub. No.: US 2005/0165413 A1
(43) Pub. Date: Jul. 28, 2005

(54) Ophthalmic microfiducial device and method for use

(76) Inventors: Stanley R. Conston, San Carlos, CA (US); Ronald K. Yamamoto, San Francisco, CA (US)

Correspondence Address: GREGORY SMITH & ASSOCIATES, 3900 NEWPARK MALL ROAD, 3RD FLOOR, NEWARK, CA, 94560, US

(21) Appl. No.: 10/507,943

(22) PCT Filed: Mar. 21, 2003
(86) PCT No.: PCT/US03/08866

Related U.S. Application Data
(60) Provisional application No. 60/366,852, filed on Mar. 22, 2002.

(51) Int. Cl. A61B 19/00

(52) U.S. Cl. 606/130

(57) ABSTRACT

This invention is directed at an ophthalmic microfiducial device that may be externally placed on the eye of the patient, the fiducial device being visible by an imaging system, and providing an external point of reference to the internal location of the anatomic structures being surgically accessed. The invention also comprises devices and methods for placing and/or removing the microfudical devices on the eye of a patient.

### FIELD OF THE INVENTION

This invention is directed at an ophthalmic microfiducial device that may be externally placed on the eye of the patient, the fiducial device being visible by an imaging system, and providing an external point of reference to the internal location of the anatomic structures being surgically accessed.

### BACKGROUND OF THE INVENTION

Glaucoma is a disease condition of the eye in which increased intraocular pressure (IOP) is created by blockage of the drainage mechanism for the aqueous fluid produced in the anterior portion of the eye. Such conditions are usually treated by topical drugs in the form of eye drops, but may result in surgical treatment if drug treatment becomes ineffective or if patient compliance is an issue. Traditional glaucoma surgery, known as a trabeculectomy, involves dissection of the eye and the forming of new holes through the trabecular meshwork portion of the drainage pathway. Although effective for a short period, long-term follow-up of these treatments shows marked increases in intraocular pressure and therefore low success rates. The procedure also involves surgical complications, such as infection, over time. Recently developed surgical treatments for glaucoma of the eye have focused on the drainage system and are approached through intrascleral incisions. These procedures are known as “non-penetrating” procedures because they do not access tissues through the cornea and visual axis structures. Viscocanalostomy and deep sclerectomy are two such procedures. These surgical procedures, as well as traditional glaucoma procedures can be improved through the combined use of high-resolution imaging and minimally invasive surgical techniques to provide treatment with fewer complications and greater repeatability.

Two high-resolution imaging modalities are currently being used to image the fine structures of the eye, primarily the anterior portion of the eye. High frequency ultrasound (HFU) and optical coherence tomography (OCT) are imaging techniques that can provide detailed anatomic information for the diagnoses, surgical treatment and post-operative follow-up, of the drainage system of the eye. Particularly, the precise location of Schlemm's Canal can be determined, in terms of radial distance from the Anterior Angle and the depth of the Canal from the surface of the eye. However, surgical intervention still requires a moderately large incision in order to access the Canal, as the externally viewed anatomic markers are difficult to relate to the precise internal location of the target tissues.

This invention is directed at an ophthalmic microfiducial device that may be externally placed on the eye of the patient, the fiducial device being visible by an imaging system and also visually by surgical microscopy, and providing an external point of reference to the internal location of the anatomic structures being surgically accessed.

### KNOWN PRIOR ART

(WO 01/10324) SPINAL FIDUCIAL IMPLANT AND METHOD
(WO 01/10302) BIODEGRADABLE SPINAL FIDUCIAL IMPLANT AND METHOD
(WO 99/26540) SURGICAL TEMPLATE ASSEMBLY AND METHOD FOR DRILLING AND INSTALLING DENTAL IMPLANTS
U.S. Pat. No. 6,096,048 NONINVASIVE, REATTACHABLE SKULL FIDUCIAL MARKER SYSTEM

### BRIEF DESCRIPTION OF THE DRAWINGS

FIG. 1 shows a first embodiment of a microfiducial device having a spherical head and a tapered spike.

FIG. 2 shows a second embodiment of a microfiducial device having a disc-shaped head as a tapered spike.

FIG. 3 shows a third embodiment of a microfiducial device comprising a chisel-shaped spike.

FIG. 4 shows a fourth embodiment of a microfiducial device comprising a faceted spike.

FIG. 5 shows a fifth embodiment of a microfiducial device having a tubular portion containing an imaging contrast agent.

FIGS. 6-8 show a device for placing and/or removing a microfiducial device from a patients eye.

### DESCRIPTION OF THE INVENTION

The invention comprises a microfiducial device for the eye, which is used to provide a stable reference point for determining operative targets from preoperative or intraoperative images. The device is designed to be placed onto the surface of the eye or inserted into the sclera without penetrating the intraocular space of the globe, and is able to be safely removed post-operatively. The device is formed of a material that can be imaged through ultrasonic and/or optical means. The invention further describes microfiducial device handling tools to be used to precisely place the device in the tissues and to retrieve it post-operatively.

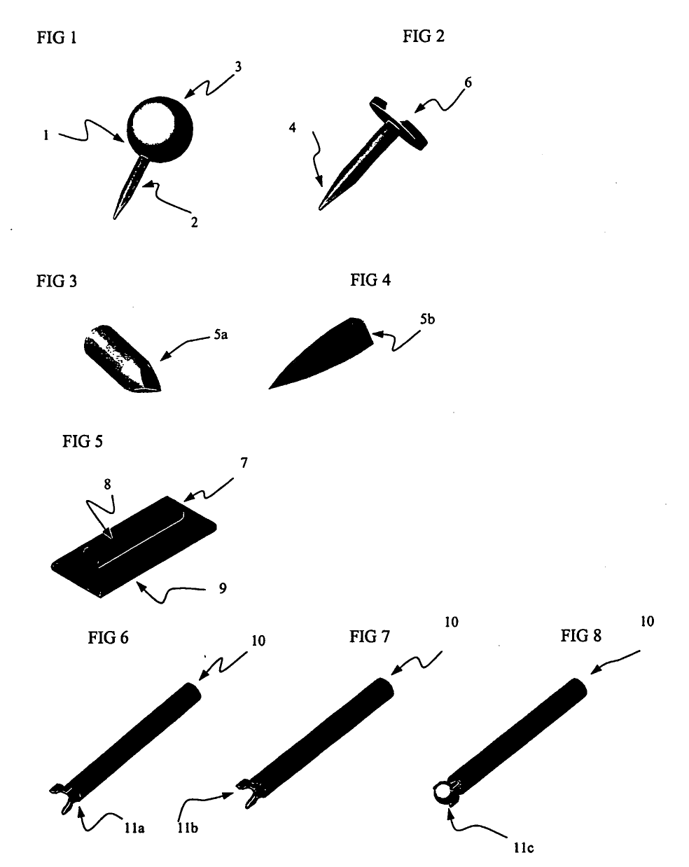

In one embodiment shown in FIG. 1, the microfiducial device 1 is in the form of a tapered spike section 2 and a spherical or hemispherical head section 3. The spike section may comprise a spike in the shape of a tapered tip 4 as shown in FIG. 2 or faceted tip 5a, 5b, as shown in FIGS. 3 and 4 and is sharp enough to allow easy entry into scleral tissues. The spike section is preferably on the order of 250-750μ in length and 50-200μ in maximum diameter or cord length. It is preferable that the spike section be long enough to provide adequate anchorage in the sclera, but not be so long as to penetrate the inner membrane of the eye. The spike section may be comprised of materials such as stainless steel, Nitinol, tungsten, polymer or ceramic. The spherical section is preferably 50-250μ in diameter. The head section may be comprised of stainless steel, Nitinol, tungsten, gold, silver or platinum to provide a high visibility contrast target under ultrasound imaging. For optical imaging the contrast target is comprised of a material with different optical absorption characteristics than the target tissues. The contrast target material may be a material with distinct optical characteristics, a dye selected for high contrast with tissues, or a dye incorporated into a material such as a polymer. Also, the device may be partly or wholly comprised of a magnetic material such that retrieval may be accomplished using a magnet passing over the surface of the eye. It is preferred that the microfiducial device provides a small precision contrast target to index relative distances of internal eye structures, with minimal obstruction or shadowing of internal tissues during imaging.

In another embodiment shown in FIG. 2, the microfiducial device is equivalent to the above with the exception that the head section is in the form of a flat disc 6, similar to a tack or nail. The disc is preferably on the order of 25-100μ thick and 50-250μ in diameter. Other shapes may be used such as hemispheres, polygons, arrow shapes, etc.

In another embodiment, only the spike section comprises the device for example, as shown in FIGS. 3 and 4. The aspects of the spike only device are as described by the spike section in the first embodiment, above.

In a further embodiment, the spike or head portion may also have a thread or wire portion attached to the proximal end. This thread portion may be incorporated to assist in holding the device in a delivery system, as well as for ease of retrieval by mechanical or manual means. The thread or wire may further comprise a severable link, such that the thread can be used during implantation, and then removed prior to imaging, in order that the thread does not interfere with imaging results. Such a severable link may be comprised of an adhesive designed to hold only to a certain tension force and then break the bond between the thread and the microfiducial device. In another embodiment, the link may be severable through electrolytic means.

Another embodiment of the invention shown in FIG. 5 incorporates a contrast target comprising a material which can be imaged through ultrasonic and/or optical means and can be placed securely on the surface of the eye, preferably with an adhesive. The microfiducial device 7 in this case may contain one or more spaces within a housing such as a section of microtubing 8, containing a contrast material such as a gas for ultrasound imaging or a dye for optical imaging. Alternatively, the housing material may be comprised of a foam, incorporating many high contrast targets such as small air or gas bubbles for ultrasound, or a dye for optical imaging. The housing may be adhered directly to the surface of the eye using contact adhesives such as medical grade acrylic adhesives or by being encapsulated into a soft, tacky material such as gelatin or a hydrogel. Alternatively, the contrast target material may be incorporated into a small polymer strip 9 that adheres to the eye in a similar manner. In one embodiment, two or more contrast targets may be incorporated onto a single polymer strip to aid three-dimensional spatial referencing of internal tissue sites. A small target of highly reflective material such as gold or steel may also be used as a surface marker or contrast target. Such a device may be in the form of a rod, tube, disc or other shape as may be required.

Preferably the device maintains a low profile on the surface of the eye so as not to interfere with access of the imaging means or surgical procedure.

Another aspect of the invention relates to the tools required to place and retrieve the microfiducial devices. A tool designed to easily implant the microfiducial devices may also incorporate a retrieval mechanism, or alternatively they may be separate tools. The retrieval tools may be single use or reusable devices.

The placement tool 10 may be comprised of a simple tube structure proximal end for handling and mechanical actuation and a distal tip for manipulating the microfiducial devices. In one embodiment, the manipulating tip may be a suction cup, with suction provided through the tube. The suction tip is particularly applicable to holding the spherical or disc-shaped implant head in a head/spike device configuration. The manipulating tip may be comprised of three or four prongs 11a, configured as bent finger-like members, as shown in FIGS. 6-8. When retracted into the tube, the prongs are compressed radially inward 11b, grabbing the head or proximal end of the microfiducial 11c. When extended, the prongs release the microfiducial device. Such configurations may be used for both placement and retrieval.

In another embodiment, the placement tool is a simple tube, wherein a microfiducial device which contains a thread or wire portion, may be delivered. The device is loaded into the tube such that the thread portion is disposed through the tube and extends beyond the proximal end. Maintaining the thread under tension will secure the device at the distal tip of the tool. The device placement is performed by inserting the spike portion into the tissues, the thread is released from the tube, and the tube discarded. The thread can be used to remove the device post-operatively.

In use, the microfiducial device is placed onto or into the surface of the eye approximately 2-3 mm radially outward from the visual limbus (the externally seen junction between the cornea and the sclera). The desired imaging modality is used to scan the eye to determine the precise location of the anatomic structures of the Anterior Chamber, such as Schlemm's Canal, the Trabecular Meshwork, Decemet's Membrane, Ciliary Body and the anterior angle. The location of the image of the microfiducial device is referenced to the location of the surgical target. During and after imaging, the surgeon has a clear external marker to provide a reference point to access the target through a minimally invasive surgical approach. Two of more microfiducial devices may be used to properly locate specific tissue regions. After the surgical target has been located, the microfiducial devices may be removed.

More than one microfiducial device may be used to provide triangulation coordinates for guided surgical intervention. Multiple markings may involve the use of the same style of microfiducial device or a combination of two or more styles as detailed above. Multiple markers are advantageous when using polar coordinates to describe the three dimensional anatomic location of various structures of the eye.

### EXAMPLES

Microfiducial devices were fabricated and tested in ex-vivo human eyes. The imaging was performed using a high frequency ultrasound scanning system operating at a center frequency of approximately 60 MHz. Microfiducial devices were fabricated from different materials at different sizes. The microfiducial devices were manually placed into the sclera of the test tissue, approximately 2-3 mm radially outward from the visual limbus. Images were recorded of each device showing the microfiducial device and the underlying tissues of the anterior angle of the eye.

The first set of microfiducial devices was comprised of gold spheres adhesively bonded to spike tips. 1 and 2 mil (1 and 2 thousandths of an inch diameter) gold wire was placed in a butane flame to melt the gold into a spherical ball. Spheres of size range from 370 to 400μ were produced. A short segment of the gold wire was left attached to the sphere for ease of handling. Ophthalmic suture needles were used to fabricate the spike sections. The distal 0.5 mm of the stainless steel needles was removed using flush cutting wire shears. The proximal cut face was honed flat using a fine sapphire stone. Two styles of needle were used, taper point and side-cutting lancet point. The taper point needle was 100μ in diameter and the lancet point was 140μ on the long axis. The gold spheres were bonded to the spikes using reinforced cyanoacrylate cement. Another set of microfiducial devices were produced using only the stainless steel spike portion without a sphere attached. Another set of microfiducial devices was produced from nickel-titanium (Nitinol) superelastic wire. The starting material was “as-drawn” condition and approximately 100μ (0.004″) diameter. The wire was treated on a fixture to hold 4 inch sections of the wire in a straight orientation. The wire was fired at 500° C. for 5 minutes and then quenched in cold water. The resultant superelastic wire was used to fabricate spike sections. The end of a wire was carefully ground using a fine carburundum grinding wheel into a tapered tip. The distal 0.5 mm of the tip was cut off and honed in a manner similar to the suture needles described above. As in the previous example, devices with and without gold spheres were fabricated.

A microfiducial device was produced using only a gold sphere. A gold sphere of 70μ diameter was produced. No spike was attached to the sphere, and a small piece of wire remained attached to be able to handle the device.

Four microfiducial devices were imaged using the high frequency ultrasound scanner. Both spike only and sphere configurations were tested. The spike test samples consisted of one stainless steel spike (from a lancet tip needle) and one Nitinol taper point spike. The other test samples consisted of a 400μ gold sphere and lancet tip spike, and the 70μ gold sphere which was placed on the surface of the sclera. All of the microfiducial devices were able to be imaged under ultrasound imaging. The implanted spikes could also be imaged under the appropriate scanning geometry, and the distal tip of each device was well clear of the inner surface of the sclera. The images showed the location of the microfiducial devices as well as the underlying tissue structures of the anterior angle of the eye.

In another experiment, surface adherent microfiducial devices were fabricated and imaged. Two styles of microfiducial devices were fabricated. Gold wire, 0.001″ diameter by 2 mm long and polyimide tubing, 0.0045″ ID×0.0056″ OD were used. The polyimide tubing was cut approximately 2 mm long and the ends sealed with cyanoacrylate adhesive. The microfiducial devices were placed into drops of a 5% solution of 250 Bloom gelatin and allowed to dry on a Teflon plate. The dried gelatin films containing the microfiducial devices were removed from the plate, and trimmed roughly rectangular. An ex-vivo human eye was placed within a fluid filled cup for ultrasonic imaging. Examples of the devices were placed manually onto the scleral surface away from the limbus. The microfiducial devices were able to be imaged, demonstrating a high contrast target on the surface of the eye, while providing imaging of the underlying tissues of the eye near the anterior angle.

1. A microfiducial ophthalmic device for use with imaging to establish a reference point on the surface of the eye for surgical intervention into tissues within the eye, comprising a contrast target.

2. The microfiducial device of claim 1 wherein the contrast target comprises a metal, polymer, or ceramic material.

3. The microfiducial device of claim 2 wherein the metal comprises gold, steel, or nickel-titanium alloy.

4. The microfiducial device of claim 2 comprising a head portion and a spike portion.

5. The microfiducial device of claim 2 comprising a spike portion.

6. The microfiducial device of claim 4 wherein the head portion is a sphere, disc, hemisphere, or polygon.

7. The microfiducial device of claim 4 wherein the spike portion is a tapered cone or multi-faceted tip.

8. The microfiducial device of claim 4 wherein the head portion and the spike portion are two different materials.

9. The microfiducial device of claim 4 wherein the head portion is between 50 and 500μ in diameter.

10. The microfiducial device of claim 4 wherein the head portion is between 50 and 150μ in diameter.

11. The microfiducial device of claim 4 wherein the spike portion is between 50 and 250μ in diameter or major cross-sectional axis.

12. The microfiducial device of claim 4 wherein the spike portion is between 50 and 750μ in length.

13. The microfiducial device of claim 1 comprising a contrast target placed directly on the surface of the eye and held by means of an adhesive, sticky or tacky substance.

14. The microfiducial device of claim 1 wherein the contrast target comprises air or gas.

15. The microfiducial device of claim 1 wherein the contrast target comprises metal.

16. The microfiducial device of claim 1 wherein the contrast target comprises an optically absorptive material.

17. The microfiducial device of claim 16 wherein the contrast target comprises a material with optically absorption characteristics distinct from tissues of the eye.

18. The microfiducial device of claim 13 wherein the contrast target is in the form of a rod, cone, tube, disc, polygon or sphere.

19. The microfiducial device of claim 13 wherein the contrast target is between 30 and 300μ in short axis or diameter.

20. The microfiducial device of claim 13 wherein the contrast target is adhered directly to the eye using an adhesive material.

21. The microfiducial device of claim 13 wherein the contrast target is contained within a thin section of material acting as a carrier.

22. The microfiducial device of claim 21 wherein the carrier material is adhered directly to the eye using an adhesive material.

23. The microfiducial device of claim 21 wherein the carrier material is a hydrogel which is adherent to the surface of the eye.

24. The microfiducial device of claim 13 wherein the device comprises two or more contrast targets.

25. An apparatus for placing a microfiducial device of claim 1 on or within the eye comprising a tube-like body with a distal end and a proximal end.

26. The apparatus of claim 25 wherein the distal end comprises a suction device.

27. The apparatus of claim 25 wherein the distal end comprises a grasping device.

28. A method for locating anatomic features in the Anterior portion of the eye by using microfiducial devices placed on or in the surface of the eye, which are imageable, to provide a common, stable point of reference for internal operative targets.

29. The method of claim 28 wherein the imaging of microfiducial devices and the internal tissues of the eye is used to locate the optimal external areas for minimally invasive surgical access to internal operative targets.

30. The method of claim 29 wherein the operative targets comprise Schlemm's Canal, the trabecular meshwork, Descement's Membrane or the ciliary body of the eye.
